(19) United States
(12) Patent Application Publication
(10) Pub. No.: US 2007/0031284 A1
(43) Pub. Date: Feb. 8, 2007

(54) Oxygen reduction system for nuclear power plant boiler feedwater and method thereof

(76) Inventor: A. John Speranza, West Hartford, CT (US)

Correspondence Address: PROTON ENERGY SYSTEM, 10 TECHNOLOGY DRIVE, WALLINGFORD, CT, 06492, US

(21) Appl. No.: 11/196,554

(22) Filed: Aug. 3, 2005

(51) Int. Cl. G01N 33/00

(52) U.S. Cl. 422/62

(57) ABSTRACT

The present invention relates to an oxygen reduction system for a boiling water reactor. In the first embodiment, the system includes an oxygen content monitor having a sensor for measuring the content of oxygen in a water stream and a controller. The controller generating a signal indicative of the measured oxygen content. A hydrogen generator having a controller is electrically coupled to the oxygen content monitor. The generator controller changes the output of the hydrogen generator in response to a signal from the oxygen content monitor.

### BACKGROUND OF THE INVENTION

1. Field of Invention

The present invention relates to an apparatus and method of operating an oxygen reduction system in a nuclear power plant and in particular to an apparatus and method of suppressing the level of oxygen in the boiler feed-water loop to minimize the occurrence of stress corrosion cracking.

2. Brief Description of the Prior Art

In general, primary cooling water is circulated to and from a reactor vessel through a turbine and condenser in a boiling reactor of a light-water nuclear power reactor. During this operation, a portion of the water in the boiler feed-water loop is converted into hydrogen gas and oxygen gas by radiation emitted during operation of the atomic reactor. The oxygen that remains as dissolved gas in the water loop can lead to the development of intergranular stress corrosion cracking (“IGSCC”) in the reactor components.

The injection of hydrogen into reactor water is utilized in boiling water reactors as one of the measures for preventing occurrence of IGSCC in metallic component material of nuclear reactor components that are in contact with the reactor water. These components include the reactor pressure vessel, reactor internal components, and piping. There is an electrochemical corrosion potential (“ECP”) of a metallic component material that results in IGSCC. It is generally believed that the potential for IGSCC increases as the ECP exceeds a critical value. In the case of stainless steels used in reactor components, the critical value of ECP is about −230 mV. The injection of hydrogen into the reactor system results in a decrease in the ECP of the metallic component materials.

Different methods of injecting hydrogen to lower the ECP have been implemented. One solution is to inject a noble metal such as platinum, rhodium, or palladium along with hydrogen gas into the reactor water. The injected noble metal deposits on the internal surfaces of the reactor components, and acts as a catalyst to recombine the oxygen and hydrogen and form water molecules. As a result, the amount of oxygen is decreased, and the ECP of the reactor components is decreased lower than the critical value.

Presently, the injection of hydrogen into reactor water is widely applied to boiling water reactors as a measure for preventing the occurrence of IGSCC. Since a large amount of hydrogen is necessary to decrease the ECP level below the critical value, reactor operators must purchase receive and store large quantities of hydrogen gas either in a compressed form in cylinders or as liquid. The purchasing, receipt and storage of a large quantity of a flammable gas is both a logistic and security issue that reactor operators must accommodate in their operations. Additionally, a typical reactor plants manually inject the hydrogen into the reactor water through a manifold located adjacent to the hydrogen cylinder storage.

Accordingly, it is considered advantageous and there is a need to provide a system and method for injecting hydrogen gas into reactor boiler feed-water in a manner that automatically adjusts for changes in the oxygen levels in the boiler feed-water. It is further considered desirable and advantageous to provide a system and method for minimizing the storage of hydrogen gas while providing sufficient quantities of hydrogen gas as operational needs demand.

### SUMMARY OF THE INVENTION

The present invention relates to an oxygen reduction system for a boiling water reactor. In the first embodiment, the system includes an oxygen content monitor having a sensor for measuring the content of oxygen in a water stream and a controller. The controller generating a signal indicative of the measured oxygen content. A hydrogen generator having a controller is electrically coupled to the oxygen content monitor. The generator controller changes the output of the hydrogen generator in response to a signal from the oxygen content monitor.

An alternate embodiment of the oxygen reduction system for a boiling water reactor includes an oxygen content monitor having a sensor for measuring the content of oxygen in a water stream and a controller. The monitor controller generates a signal indicative of oxygen content. A hydrogen generator having a hydrogen gas output and a controller is electrically coupled to the oxygen content monitor. The generator controller changes the output of hydrogen generator in response to a signal from oxygen content monitor. A storage tank is selectively coupled to the hydrogen generator to receive hydrogen gas when the oxygen content signal is indicative of an oxygen content below a first threshold.

A method for reducing oxygen in a water is also provided. First the method measures the content of oxygen in the water. A signal is generated indicative of the level of the oxygen content. A determination is made if the oxygen content is greater than or equal to a first threshold. Hydrogen gas is generated at a first output if the oxygen content is greater than the first threshold and, the hydrogen gas is injected into said water. The hydrogen gas required to reduce oxygen content is determined when the oxygen content is above the first threshold. If the amount of hydrogen gas generated is increased to equal the amount of hydrogen gas required if the hydrogen gas generation is less than the amount of hydrogen gas required.

Other features and advantages of the present invention will be apparent from the following detailed description of the invention and the accompanying drawings in which the same numerals refer to like elements.

### BRIEF DESCRIPTION OF THE DRAWINGS

FIG. 1 is a schematic diagram illustrating the system structure of a first embodiment of the present invention;

FIG. 2 is a schematic diagram illustrating the system structure of a second embodiment of the present invention;

FIG. 3 is a flow diagram illustrating the method of reducing oxygen content in the system structure shown in FIG. 1; and,

FIG. 4 is a flow diagram illustrating the method of reducing oxygen content in the system structure shown in FIG. 2.

### DETAILED DESCRIPTION

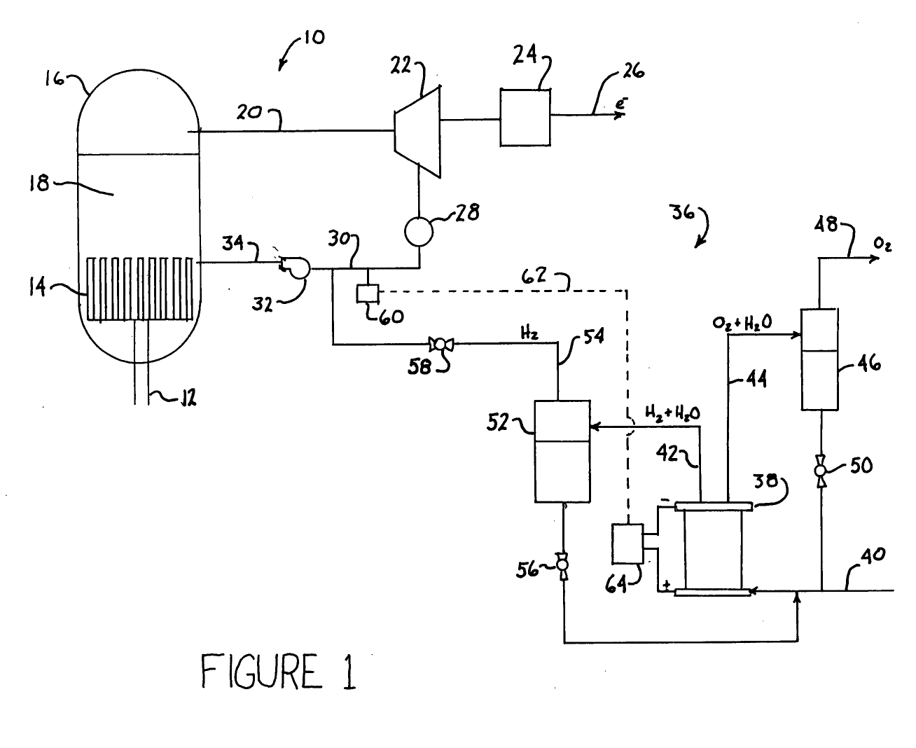

Nuclear power plants utilize radioactive material to generate heat to boil water. A typical boiler feed-water reactor (“BWR”) 10 is shown in FIG. 1. The BWR 10 uses control rods 12 and fuel rods 14 to produce a nuclear reaction in the reactor vessel 16. The nuclear reaction produces heat and radioactivity which boils water 18 in the vessel 16 to produce steam. The steam exits the vessel 16 through conduit 20 where it flows to drive a turbine 22. The rotational energy of the turbine in turn drives a generator 24 to produce electricity 26.

After exiting the turbine 22, the steam condenses in condensator 28. The condensed feed-water then proceeds via conduit 30 to pump 32. The pump 32 returns the feed-water back into the reactor vessel 16 via conduit 34.

Since the water is subjected to radiation, at least some portion of the feed-water will be converted into oxygen gas. To avoid the problems of a ECP, the preferred embodiment incorporates a hydrogen generation system 36 to inject hydrogen gas into the feed-water to reduce the oxygen content. The hydrogen generator 36 includes a hydrogen conversion device 38 that receives a fuel via conduit 40. The hydrogen conversion device 38 disassociates hydrogen atoms from the fuel to produce hydrogen gas that exits the hydrogen conversion device 38 through conduit 42. In the preferred embodiment, the hydrogen conversion device is an electrochemical cell stack and the fuel is water. The electrochemical cell stack disassociates the hydrogen from water through electrolysis resulting in the creation of hydrogen and oxygen gas. Preferably, the electrochemical cell will be an ion-conducting polymer membrane electrode type cell that includes an anode and a cathode electrodes that contain a noble metal, with the electrodes being separated by a solid polymer membrane. In the preferred embodiment, the noble metals used in the electrodes will include platinum. The electrochemical cell stack may consist of a single cell having the anode and cathode chambers separated by the polymer electrode membrane, or may be comprised of a plurality cells each having an anode and cathode chamber and being arranged electrically in series or in parallel.

It should be appreciated that the electrochemical cell may also be any other suitable electrochemical cell such as, but not limited to, alkaline, phosphoric acid, or solid oxide based cells. The hydrogen conversion device may also be any non-electrochemical system capable of producing hydrogen gas such as, but not limited to, a steam methane reformation, natural gas reformation, coal reformation, hydrocarbon reformation, partial oxidation reactors, ceramic membrane reactor, photolysis, photoelectrolysis, photochemical reactors, photobiological reactors, anaerobic digesters or bio-mass gasification.

A multi-phase mixture of oxygen gas and water exits the hydrogen conversion device 38 via conduit 44 and enters a oxygen water phase separator 46. The separator 46 liberates the oxygen gas from the water through a pressure drop which results in the water falling to the bottom of the separator under gravity. The separated oxygen gas exits the phase-separator 46 via conduit 48 and is vented to the atmosphere, or retained by the plant operator for further use. The water is periodically drained from the phase separator 46 by the opening of valve 50 that connects the phase-separator 46 to conduit 40.

Hydrogen gas exits the hydrogen conversion device 38 via conduit 42 entrained in a small amount of water that permeates across the polymer membrane. The water is removed from the hydrogen gas in hydrogen-water phase separator 52. In a similar manner to separator 46, due to a pressure drop in the separator 52, the water drops under gravity to the bottom of the separator 52 while the gas exits via conduit 54. Water is periodically drained from the separator 52 by valve 56 which connects the separator 52 with conduit 40. Hydrogen gas flows through check valve 58 and enters the feed-water conduit 30. It should be appreciated that additional valves, regulators, fittings and vent conduits familiar to those skilled in the art may be utilized with the present invention, but have been omitted for the sake of clarity.

An oxygen monitor 60 is coupled to conduit 30 for measuring the oxygen content of the feed-water. The monitor 60 may be of any suitable type, such as Instrument Model SM31 manufactured by the EXA Corporation. The monitor 60 transmits a signal indicative of the oxygen content in the reactor feed-water via line 62 to controller 64. The controller 64 receives the signal and adjusts the output of the hydrogen conversion device 38 to compensate for the varying oxygen content in the feed-water. In the preferred embodiment, the controller 64 either increases or decreases current provided to the cell stack by a power supply to increase and decrease the hydrogen production. It should be appreciated that the control methodology may be implemented in any suitable manner for a microprocessor or analog control system such as, but not limited to, look-up tables, databases, and algorithms. It is contemplated that the controller 64 may include fuzzy-logic other heuristic algorithms that allow the hydrogen generation system 36 to predict and adjust the hydrogen requirements derived from historical data of the BWR's reaction to the injection of hydrogen.

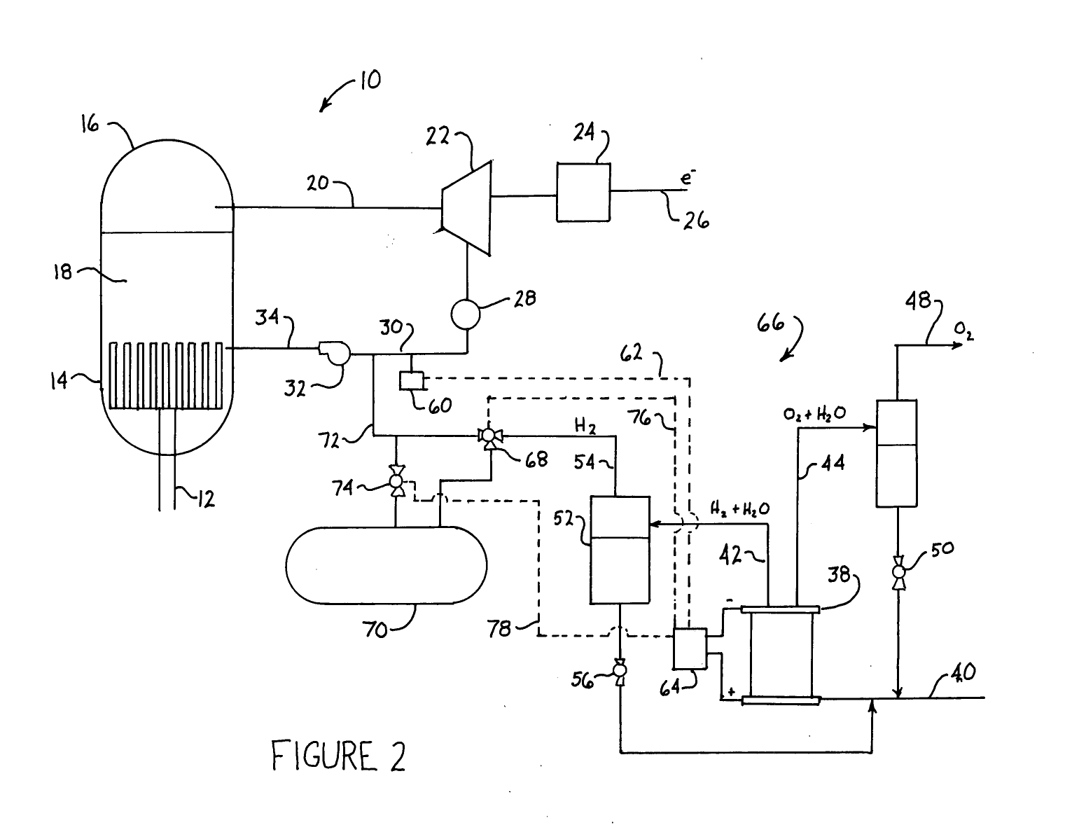

An alternate embodiment hydrogen generation system 66 is shown in FIG. 2. In this embodiment, a three-way valve 68 is coupled to conduit 54. This valve 68 allows the diverting of hydrogen gas in response to a signal 76 from controller 64. When the hydrogen generation system 66 has extra capacity, or the oxygen content in the reactor feed-water is low, controller 64 transmits a signal to valve 68 to divert hydrogen gas to storage tank 70. The storage tank 70 is coupled to conduit 72 through solenoid valve 74. A signal 78 from the controller 64 opens the valve 74 allowing hydrogen gas from the storage tank 70 to be injected into the conduit 30. This embodiment allows the reactor operator to utilize excess capacity to build an alternate supply of hydrogen gas that may be utilized when extra capacity is required, or if the hydrogen generation system is offline for maintenance. This embodiment my also allow the utilization of a smaller hydrogen generation system that runs continuously at a constant rate and utilizing the storage tank 70 to level the loading requirements on the generation system 66.

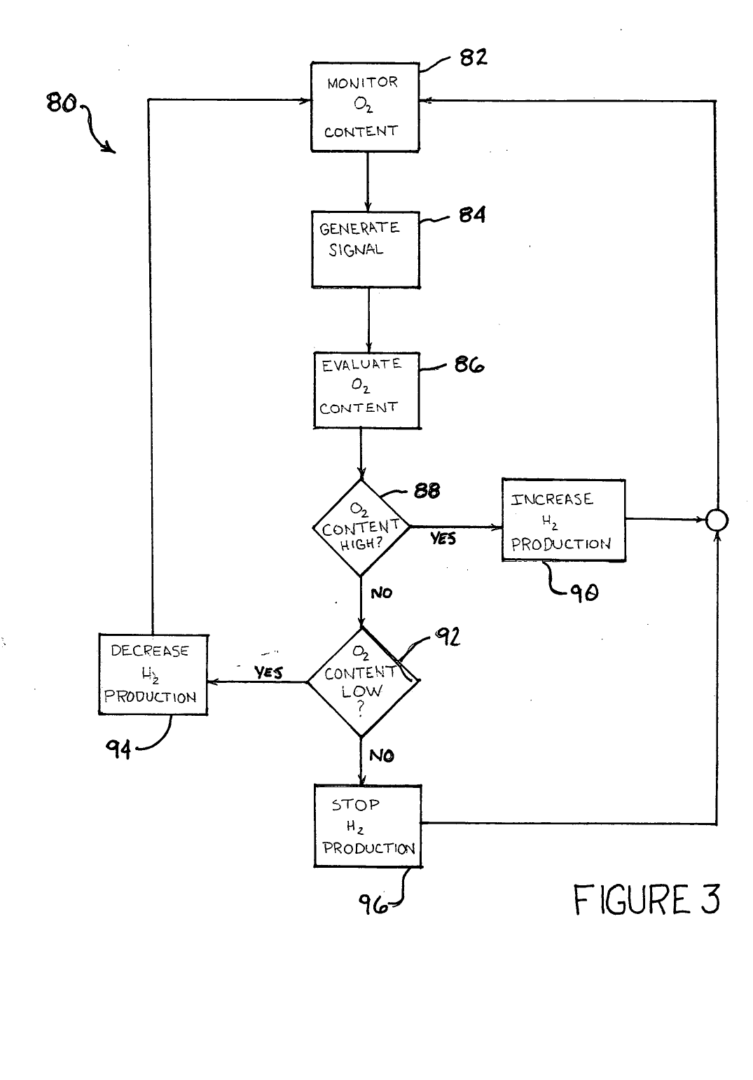

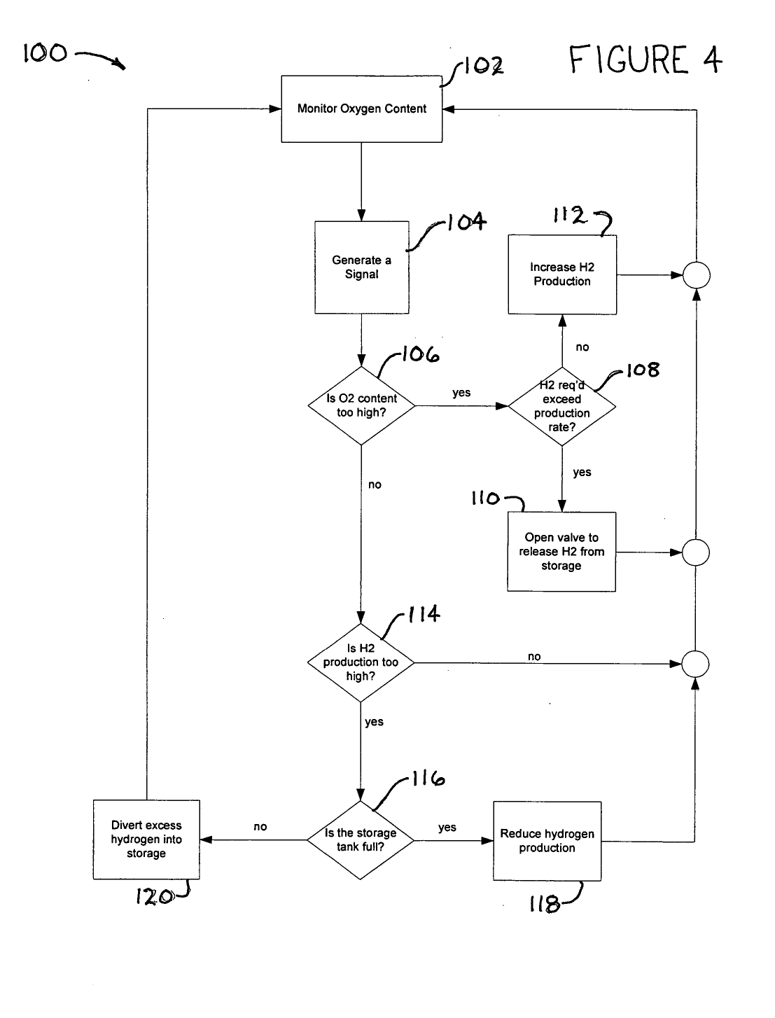

FIGS. 3 and 4 are flow diagrams depicting the operation of the generating system 36, 66. These methods may be included and executed in the controller application code in one or more of the individual components of the system 36, 66, or may be embodied in a single central controller (not shown). These methods are embodied in computer instructions written to be executed by a microprocessor typically in the form of software. The software can be encoded in any language, including, but not limited to, assembly language, VHDL (Verilog Hardware Description Language), VHSIC HDL (Very High Speed IC Hardware Description Language), Fortran (formula translation), C, C++, Visual C++, Java, ALGOL (algorithmic language), BASIC (beginners all-purpose symbolic instruction code), visual BASIC, ActiveX, HTML (HyperText Markup Language), and any combination or derivative of at least one of the foregoing. Additionally, an operator can use an existing software application such as a spreadsheet or database and correlate various components enumerated in the algorithms. Furthermore, the software can be independent of other software or dependent upon other software, such as in the form of integrated software.

Referring to FIG. 3 an oxygen reduction system control method 80 of FIG. 1 will now be described. Method 80 starts at block 82 where the oxygen content sensor 60 transmits a signal 84 representative of the level of oxygen in the reactor feed-water to controller 64. The method 80 proceeds to block 86 where the oxygen content is evaluated. If the oxygen content is determined to be too high, the method proceeds to block 90 to increase hydrogen production. In the preferred embodiment, the hydrogen production accomplished by adjusting the output of a power supply to increase or decrease the electrical current provided to the electrochemical cell stack. After adjusting the hydrogen production, method 80 then proceeds to loop back to block 82.

If the oxygen content is not too high, then method 80 evaluates in decision block 92 if the oxygen content is too low for the amount of hydrogen being produced. Since excess hydrogen is undesirable, the method 80 decreases in block 94 the amount of hydrogen being produced and proceeds to loop back to block 82.

Finally, if the oxygen content falls below a predetermined threshold level, hydrogen production is stopped in block 96. The method 80 then loops back to block 82 to continue monitoring of the oxygen content. It should be appreciated that the method 80 would be expected to operate continuously while the BWR is in operation.

Referring to FIG. 4, an oxygen reduction system control method 100 of FIG. 2 will now be described. Method 100 starts at block 102 where the oxygen content sensor 60 measures the oxygen level and transmits a signal 104 to the controller 64. The method 100 then proceeds to evaluate 106 the level of oxygen in the reactor feed-water. If the level is too high, the method 100 determines in block 108 whether the amount of hydrogen required to mitigate the oxygen exceeds the production rate of the generation system 66. If the level exceeds production capacity, the method 100 proceeds to block 110 where the valve 74 is opened allowing stored hydrogen to supplement the produced hydrogen and the method loops back to block 102. If method 100 determines in block 108 that the required hydrogen does not exceed the generation system 66 capacity, the method 100 proceeds to block 112 where hydrogen production is increased and the method 100 loops back to block 102.

If the method 100 determines in block 106 that the oxygen content is not too high for the level of hydrogen being injected, the method 100 proceeds to block 114 to determine if the hydrogen production is too high. If the hydrogen production rate is not too high, the method 100 loops back to block 102. If the method 100 determines 116 that the production rate is too high for the level of oxygen in the reactor feed-water, the method 100 then queries the amount of stored hydrogen in the storage tank 70. If the tank is full, hydrogen production is reduced in block 118 and the method 100 loops back to block 102. If the storage tank 70 is not full, the method 100 transmits a signal to valve 68 to divert excess hydrogen to the storage tank 70. The method 100 then proceeds to loop back to block 102.

While the invention has been described with reference to a preferred embodiment, it will be understood by those skilled in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiment disclosed as the best mode contemplated for carrying out this invention.

What is claimed is:

1. An oxygen reduction system for a boiling water reactor comprising:
an oxygen content monitor, said monitor having a sensor for measuring the content of oxygen in a water stream and a controller, said controller generating a signal indicative of said oxygen content;
a hydrogen generator having a controller, said generator controller being electrically coupled to said oxygen content monitor whereby said controller changes the output of said hydrogen generator in response to a signal from said oxygen content monitor.

2. The oxygen reduction system of claim 1 wherein said hydrogen generator further comprises an electrochemical cell having an anode chamber and a cathode chamber separated from one another by a membrane.

3. The oxygen reduction system of claim 2 where said membrane is made from an ion-conducting polymer having an anode and cathode electrode disposed thereon.

4. The oxygen reduction system of claim 3 wherein said electrochemical cell is comprised of a plurality of individual cells, each individual cell having an anode and cathode chamber separated by an ion conducting polymer membrane, and each individual cell being electrically arranged in series.

5. The oxygen reduction system of claim 4 wherein said anode electrode contains platinum.

6. The oxygen reduction system of claim 1 wherein said hydrogen generator is selected from a group comprising: alkaline electrochemical cells, phosphoric acid electrochemical cells, solid oxide electrochemical cells, steam methane reformer, natural gas reformer, coal reformer, hydrocarbon reformer, partial oxidation reactors, ceramic membrane reactor, photolysis reactor, photoelectrolysis reactor, photochemical reactors, photobiological reactors, anaerobic digesters or bio-mass gasification reactors.

7. The oxygen reduction system of claim 3 further comprising a power supply electrically coupled to said generator controller and said electrochemical cell whereby said hydrogen generator output is changed by increasing or decreasing the electrical current provided to said electrochemical cell from said power supply.

8. The oxygen reduction system of claim 7 further comprising a phase separator fluidly coupled to said electrochemical cell, said phase separator having a hydrogen gas output and a water output.

9. An oxygen reduction system for a boiling water reactor comprising:
an oxygen content monitor, said monitor having a sensor for measuring the content of oxygen in a water stream and a controller, said controller generating a signal indicative of said oxygen content;
a hydrogen generator having a hydrogen gas output and a controller, said generator controller being electrically coupled to said oxygen content monitor whereby said controller changes the output of said hydrogen generator in response to a signal from said oxygen content monitor; and,
a storage tank selectively coupled to said hydrogen generator to receive hydrogen gas when said oxygen content signal is indicative of a oxygen content below a first threshold.

10. The oxygen reduction system of claim 9 further comprising
a conduit fluidly coupled to said hydrogen generator and said storage tank; and,
a valve fluidly coupled between said storage tank and said conduit.

11. The oxygen reduction system of claim 10 wherein said valve is electrically coupled to said generator controller whereby said valve actuates in response to a signal from said generator controller.

12. The oxygen reduction system of claim 11 wherein said generator controller actuates said valve to an open position when said oxygen content signal is indicative of a oxygen content above a second threshold.

13. The oxygen reduction system of claim 12 further comprising a means for selectively coupling said storage tank to said hydrogen generator, said means for coupling being coupled to said conduit, said hydrogen generator and said storage tank whereby said means for coupling flows hydrogen to said conduit or said storage tank in response to a signal from said generator controller.

14. A method for reducing oxygen in a water comprising the steps of:
measuring the content of oxygen in said water;
generating a signal indicative of the level of said oxygen content;
determining if the oxygen content is greater than or equal to a first threshold;
generating hydrogen gas at a first output if said oxygen content is greater than said first threshold; and,
injecting said hydrogen gas into said water.

15. The method of reducing oxygen in water of claim 14 further comprising the steps of:
determining the amount of hydrogen gas required when said oxygen content is above said first threshold;
increasing the amount of hydrogen gas generated to equal the amount of hydrogen gas required if said hydrogen gas generation is less than said amount of hydrogen gas required.

16. The method for reducing oxygen in water of claim 15 further comprising the step of decreasing the amount of hydrogen gas generated if said hydrogen gas generation is greater than said amount of hydrogen gas required.

17. The method for reducing oxygen in water of claim 15 further comprising the step of storing hydrogen gas when said hydrogen gas generated is greater than said amount of gas required.

18. The method for reducing oxygen in water of claim 17 further comprising the step of releasing said stored hydrogen gas when said oxygen content is above a second threshold.

19. The method for reducing oxygen in water of claim 18 further comprising the step of selectively diverting a portion of said hydrogen gas generated to a storage tank if said oxygen content is above said first threshold and below said second threshold.
